(19) United States
(12) Patent Application Publication
(10) Pub. No.: US 2012/0305497 A1
(43) Pub. Date: Dec. 6, 2012

(54) Fenton and Fenton-Like System Hardening Agent and Usage Thereof

(75) Inventors: Jun Ma, Harbin (CN); Liwei Chen, Harbin (CN); Xuchun Li, Harbin (CN); Jing Zhang, Harbin (CN); Yinghong Guan, Harbin (CN); Jingyun Fang, Harbin (CN)

(73) Assignee: HARBIN INSTITUTE OF TECHNOLOGY, Harbin City, Heilongjiang Province (CN)

(21) Appl. No.: 13/578,244

(22) PCT Filed: Oct. 18, 2010
(86) PCT No.: PCT/CN10/77817

§ 371 Date: Aug. 9, 2012

(30) Foreign Application Priority Data
Feb. 10, 2010 (CN) 201010108952.7

(51) Int. Cl. C02F 1/72; C07C 211/51; C01B 21/16; C07C 239/08; C01B 17/62; C01B 21/14

(52) U.S. Cl. 210/759; 423/519.2; 423/386; 423/388; 423/407; 564/301; 564/305; 252/181

(57) ABSTRACT

Fenton and Fenton-like system enhancing agent and the usage thereof are provided. It relates to a water treatment enhancer (enhancing agent) and the usage thereof. It widens water pH range of Fenton and Fenton-like system reaction. It reduces amount of Fe²⁺ required for Fenton reaction. It increases rate of Fenton-like reaction. The enhancing agent is selected from sodium sulfite, lithium sulfite, potassium sulfite, magnesium sulfite, calcium sulfite, hydroxylamine hydrochloride, hydroxylamine perchlorate, hydroxylamine sulfate, hydrazine, N,N-diethylhydroxylamine, amino ethanolamine, hydroxylamine solution or N,N,N′,N′-tetrasubstituted p-phenylenediamine. The method of use of enhancing agent comprises the steps of: adding Fenton or Fenton-like system enhancing agent, an agent for enhancement and hydrogen peroxide into water subject to treatment; and mixing and allowing reaction. The enhancing agent can increase the rate of reaction for the water treatment and reduce the dosage of the agent for enhancement.

### BACKGROUND OF THE PRESENT INVENTION

1. Field of Invention

The present invention relates to an enhancing agent for water treatment and its method of use.

2. Description of Related Arts

Water pollution problem has become more and more serious in our country and the complexity level in relation to water quality has become higher. Though traditional biological treatment has the advantage of low cost, the drawback is large space requirement. Accordingly, traditional biological treatment method is only suitable for treating sewage which has low concentration of pollutants and is readily biodegradable. For treating organic sewage which is highly concentrated in pollutants and difficult to degrade, the traditional biological treatment method has a relatively low efficiency. In addition, the system activation time is relatively long.

Recently, advanced oxidation technology is the hot spot of both national and international research subject. In short, advanced oxidation refers to the utilization of different systems to produce free radicals so as to decompose organic pollutants, to increase the level of biodegradation, or to achieve complete mineralization directly. Fenton and Fenton-like system is the most widely used systems of advanced oxidation and its research and development has a history of more than one century. Fenton system mainly utilizes Fe²⁺ and hydrogen peroxide (or other type of oxidant) to produce hydroxyl radicals or transient high valent iron to attack the target substance in the reaction system. Fenton-like system utilizes non-divalent iron, which includes transition metal ions (such as Fe³⁺, Mn, Cu, Co, Ni, and etc.), transition metal oxides, transition metal ions or oxides in loaded carrier which reacts with hydrogen peroxide (or other oxidant) to produce hydroxyl radicals or transient high valent iron for attacking the target subject in the reaction system.

Fenton system has the advantageous effect of fast reaction rate. However, the water pH requirement of Fenton system is very restrict. The optimal water pH of the water subject to treatment in a Fenton system is about 3. Accordingly, a large amount of acid or alkaline is required and used to pre-treat the water subject to treatment before using the Fenton system for water treatment. Thus the work load is increased and the cost is increased accordingly. In addition, because a large amount of iron is added in the Fenton system, post-treatment of the water not only requires pH adjustment, but also removal of iron ions in the water. Therefore, the application of Fenton system is limited.

Fenton-like system decreases the amount of transition metal being added and the applicable pH range is relatively broader (pH 3˜4). However, the reaction rate is much slower than that of the Fenton system and the water treatment effect is much poorer than that of the Fenton system.

At present, some researchers add Fenton complexing agents such as benzoquinone and EDTA into the Fenton system to promote Fenton reaction. However, this kind of complexing agents is organic substance in nature. While the complexing agent is capable of promoting Fenton reaction, the concentration of organic substance in the water is also increased. Therefore the use of this kind of complexing agent is not desirable for practical application.

### SUMMARY OF THE PRESENT INVENTION

An object of the present invention is to provide a Fenton and Fenton-like system enhancing agent (enhancer) and its method of use so as to solve the problems of the Fenton and Fenton-like system in which the pH of the reaction is restrictive, the quantity requirement of Fe²⁺ is excessively high and the reaction rate is low.

### Technical Solution

According to the preferred embodiment of the present invention, the Fenton and Fenton-like system enhancing agent is selected from sodium sulfite, lithium sulfite, potassium sulfite, magnesium sulfite, calcium sulfite, hydroxylamine hydrochloride, hydroxylamine perchlorate, hydroxylamine sulfate, hydrazine, N-N-diethylhydroxylamine, amino ethanolamine, hydroxylamine solution and N,N,N′,N′-tetrasubstituted p-phenylenediamine.

A method of use of the above Fenton and Fenton-like system enhancing agent comprises the steps of: adding a Fenton and Fenton-like system enhancing agent, an agent for enhancement and hydrogen peroxide into water subject to treatment, wherein the water subject to treatment contains pollutants; and mixing uniformly and allowing reaction such that an enhancement treatment for the Fenton and Fenton-like system is complete, wherein a molar ratio of the Fenton and Fenton-like system enhancing agent being added and the pollutants in the water subject to treatment is 1˜10:1˜1000, a molar ratio of the hydrogen peroxide being added and the pollutants in the water subject to treatment is 1˜100:1, and a molar ratio of the agent for enhancement being added and the pollutants in the water subject to treatment is 0.1˜1:1, wherein the agent for enhancement is selected from salt of divalent iron, salt of trivalent iron, salt of high valent iron, zerovalent iron, Fe4O3, Iron (III) oxide, Iron (III) oxide-hydroxide, iron (II) oxide, cobalt, cerium, vanadium, nickel, zinc, chromium, silver, scandium, yttrium, zirconium, niobium, molybdenum, technetium, ruthenium, rhodium, palladium, cadmium, lutetium, hafnium, tungsten, rhenium, osmium, iridium, lawrencium, CoO, Ce₂O₃, CeO₂, V₂O₅, VO₂, NiO, ZnO, Cr₂O₃, Ag₂O, Sc₂O₃, Y₂O₃, ZrO₂, Nb₂O₅, MoO₃, Tc₂O₇, RuO₂, Rh₂O₃, PdO, CdO, Lu₂O₃, HfO₂, WO₃, ReO₂, Re₂O₇, OsO₄, OsO₂, IrO₂ and Rh₂O₃.5(H₂O); wherein the Fenton and Fenton-like system enhancing agent is selected from sodium sulfite, lithium sulfite, potassium sulfite, magnesium sulfite, calcium sulfite, hydroxylamine hydrochloride, hydroxylamine perchlorate, hydroxylamine sulfate, hydrazine, N-N-diethylhydroxylamine, amino ethanolamine, hydroxylamine solution and N,N,N′,N′-tetrasubstituted p-phenylenediamine.

According to the preferred embodiment of the present invention, the Fenton and Fenton-like system enhancing agent can enhance the reaction of Fenton or Fenton-like system which utilizes the following agents (the agent for enhancement): salt of divalent iron, salt of trivalent iron, salt of high valent iron, zerovalent iron, Fe4O3, Iron (III) oxide, Iron (III) oxide-hydroxide, iron (II) oxide, cobalt, cerium, vanadium, nickel, zinc, chromium, silver, scandium, yttrium, zirconium, niobium, molybdenum, technetium, ruthenium, rhodium, palladium, cadmium, lutetium, hafnium, tungsten, rhenium, osmium, iridium, lawrencium, CoO, Ce₂O₃, CeO₂, V₂O₅, VO₂, NiO, ZnO, Cr₂O₃, Ag₂O, Sc₂O₃, Y₂O₃, ZrO₂, Nb₂O₅, MoO₃, Tc₂O₇, RuO₂, Rh₂O₃, PdO, CdO, Lu₂O₃, HfO₂, WO₃, ReO₂, Re₂O₇, OsO₄, OsO₂, IrO₂ and/or Rh₂O₃.5(H₂O). The Fenton and Fenton-like system enhancing agent of the present invention can be a single component system or a multi-component system containing two or more components for enhancing the Fenton and Fenton-like system, increasing the reaction rate of water treatment, reducing a quantity requirement of the agent for enhancement while capable of providing a wide selection range of agent for enhancements.

### Advantageous Effect

According to the preferred embodiment of the present invention, the Fenton and Fenton-like system enhancing agent does not require an adjustment of water pH to a value below 4 for carrying out the Fenton or Fenton-like reaction during the enhancement treatment. In addition, the water pH does not need to be adjusted to neutral after the Fenton or Fenton-like reaction and the water after the enhancement treatment can be arranged for post-water treatment directly.

The method of use of the Fenton and Fenton-like system enhancing agent is simple and easy to apply. There is no additional treatment equipment requirement with a wide range of applicability while the reaction temperature requirement is low. The quantity requirement of Fenton and Fenton-like system enhancing agent is low. After the Fenton and Fenton-like system enhancing agent is dissolved in the water, the water is safe to human and animals and no additional equipment or steps is required for removal.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 illustrates the test results of the comparison test in Embodiment 12, where the removal efficiency curve of pollutant in the water subject to treatment using the method in Embodiment 12 of the present invention is indicated by “▪”, while the removal efficiency curve of pollutant in the water subject to treatment using the conventional Fenton system is indicated by “□”. FIG. 2 illustrates the test results of the comparison test in Embodiment 13, where the removal efficiency curve of pollutant in the water subject to treatment using the method in Embodiment 13 of the present invention is indicated by “▪”, while the removal efficiency curve of pollutant in the water subject to treatment using the conventional Fenton system is indicated by “□”. FIG. 3 illustrates the removal efficiency of enhancement treatment in relation to water pH in Embodiment 28, where the removal rate of pollutants in the water subject to treatment using the Fenton-like system with enhancement treatment is indicated by “▪” while the removal rate of pollutants in the water subject to treatment using the Fenton system with enhancement treatment is indicated by “”.

### DETAILED DESCRIPTION OF THE PREFERRED EMBODIMENT

The embodiment of the present invention as shown and described below is exemplary only and not intended to be limiting. Therefore, this invention includes all modifications or any combination encompassed within the spirit and scope of the followings.

### Embodiment 1

The Fenton and Fenton-like system enhancing agent is selected from sodium sulfite, lithium sulfite, potassium sulfite, magnesium sulfite, calcium sulfite, hydroxylamine hydrochloride, hydroxylamine perchlorate, hydroxylamine sulfate, hydrazine, N-N-diethylhydroxylamine, amino ethanolamine, hydroxylamine solution and N,N,N′,N′-tetrasubstituted p-phenylenediamine.

According to this embodiment of the present invention, the Fenton and Fenton-like system enhancing agent can enhance the reaction of Fenton or Fenton-like system utilizing the following agents: salt of divalent iron, salt of trivalent iron, salt of high valent iron, zerovalent iron, Fe4O3, Iron (III) oxide, Iron (III) oxide-hydroxide, iron (II) oxide, cobalt, cerium, vanadium, nickel, zinc, chromium, silver, scandium, yttrium, zirconium, niobium, molybdenum, technetium, ruthenium, rhodium, palladium, cadmium, lutetium, hafnium, tungsten, rhenium, osmium, iridium, lawrencium, CoO, Ce₂O₃, CeO₂, V₂O₅, VO₂, NiO, ZnO, Cr₂O₃, Ag₂O, Sc₂O₃, Y₂O₃, ZrO₂, Nb₂O₅, MoO₃, Tc₂O₇, RuO₂, Rh₂O₃, PdO, CdO, Lu₂O₃, HfO₂, WO₃, ReO₂, Re₂O₇, OsO₄, OsO₂, IrO₂ and Rh₂O₃.5(H₂O). The Fenton and Fenton-like system enhancing agent according to this embodiment of the present invention can be a single component system or a multi-component system containing two or more components for enhancing the Fenton and Fenton-like system, increasing the reaction rate of water treatment, reducing a quantity requirement of the agent for enhancement while capable of providing a wide selection range of agent for enhancements.

According to this embodiment of the present invention, the Fenton and Fenton-like system enhancing agent does not require an adjustment of water pH to a value below 4 for carrying out the Fenton or Fenton-like reaction during the enhancement treatment. In addition, the water pH does not need to be adjusted to neutral after the Fenton or Fenton-like reaction and can be arranged for post-water treatment directly.

According to this embodiment, if the Fenton and Fenton-like system enhancing agent includes two or more components, the ratio of different components can be free to adjust and is not fixed.

The hydroxylamine solution of this embodiment is provided by Shanghai Rui Cong Technology Development Co., Ltd.

### Embodiment 2

The embodiment 2 and the embodiment 1 have identical steps and parameters except that in the embodiment 2, the Fenton and Fenton-like system enhancing agent is further selected from one or more of the followings: ammonium sulfite, sulfite, sodium metabisulfite, lithium metabisulfite, potassium metabisulfite, magnesium metabisulfite, calcium metabisulfite, ammonium metabisulfite, sodium dithionite, lithium dithionite, potassium dithionite, magnesium dithionite, calcium dithionite, ammonium dithionite and humic substance.

According to this embodiment, the Fenton and Fenton-like system enhancing agent includes two or more components, the ratio of different components can be free to adjust and is not fixed.

### Embodiment 3

The embodiment 3 and the embodiment 1 have identical steps and parameters except that in the embodiment 3, the Fenton and Fenton-like system enhancing agent is sodium sulfite, lithium sulfite, potassium sulfite, magnesium sulfite, calcium sulfite, hydroxylamine hydrochloride, hydroxylamine perchlorate, hydroxylamine sulfate, hydrazine, N-N-diethylhydroxylamine, amino ethanolamine or N,N,N′,N′ -tetrasubstituted p-phenylenediamine.

### Embodiment 4

According to this embodiment, the Fenton and Fenton-like system enhancing agent is sodium sulfite, lithium sulfite, potassium sulfite, magnesium sulfite, calcium sulfite, hydroxylamine hydrochloride, hydroxylamine perchlorate, hydroxylamine sulfate, hydrazine, N-N-diethylhydroxylamine, isopropyl hydroxylamine, potassium permanganate, amino ethanolamine, N,N,N′,N′-tetrasubstituted p-phenylenediamine, ammonium sulfite, sulfite, sodium metabisulfite, lithium metabisulfite, potassium metabisulfite, magnesium metabisulfite, calcium metabisulfite, ammonium metabisulfite, sodium dithionite, lithium dithionite, potassium dithionite, magnesium dithionite, calcium dithionite, ammonium dithionite or humic substance.

### Embodiment 5

The Fenton and Fenton-like system enhancing agent is used by employing the following method of use which comprises the steps of: adding a Fenton and Fenton-like system enhancing agent, an agent for enhancement and hydrogen peroxide into water subject to treatment, wherein the water subject to treatment contains pollutants; and mixing uniformly and allowing reaction such that an enhancement treatment for the Fenton and Fenton-like system is complete, wherein a molar ratio of the Fenton and Fenton-like system enhancing agent being added and the pollutants in the water subject to treatment is 1˜10:1˜1000, a molar ratio of the hydrogen peroxide being added and the pollutants in the water subject to treatment is 1˜100:1, and a molar ratio of the agent for enhancement being added and the pollutants in the water subject to treatment is 0.1˜1:1, wherein the agent for enhancement is selected from salt of divalent iron, salt of trivalent iron, salt of high valent iron, zerovalent iron, Fe4O3, Iron (III) oxide, Iron (III) oxide-hydroxide, iron (II) oxide, cobalt, cerium, vanadium, nickel, zinc, chromium, silver, scandium, yttrium, zirconium, niobium, molybdenum, technetium, ruthenium, rhodium, palladium, cadmium, lutetium, hafnium, tungsten, rhenium, osmium, iridium, lawrencium, CoO, Ce₂O₃, CeO₂, V₂O₅, VO₂, NiO, ZnO, Cr₂O₃, Ag₂O, Sc₂O₃, Y₂O₃, ZrO₂, Nb₂O₅, MoO₃, Tc₂O₇, RuO₂, Rh₂O₃, PdO, CdO, Lu₂O₃, HfO₂, WO₃, ReO₂, Re₂O₇, OsO₄, OsO₂, IrO₂ and Rh₂O₃.5(H₂O); wherein the Fenton and Fenton-like system enhancing agent is selected from sodium sulfite, lithium sulfite, potassium sulfite, magnesium sulfite, calcium sulfite, hydroxylamine hydrochloride, hydroxylamine perchlorate, hydroxylamine sulfate, hydrazine, N-N-diethylhydroxylamine, amino ethanolamine, hydroxylamine solution and N,N,N′,N′-tetrasubstituted p-phenylenediamine.

According to this embodiment, the Fenton and Fenton-like system enhancing agent includes two or more components, the ratio of different components can be free to adjust and is not fixed.

According to this embodiment, the agent for enhancement includes two or more agent for enhancement components, the ratio of different agent for enhancement components can be free to adjust and is not fixed.

According to this embodiment of the present invention, the Fenton and Fenton-like system enhancing agent does not require an adjustment of water pH to a value below 4 for carrying out the Fenton or Fenton-like reaction during the enhancement treatment. In addition, the water pH does not need to be adjusted to neutral after the Fenton or Fenton-like reaction and can be arranged for post-water treatment directly. In particular, the quantity requirement of the agent for enhancement being used is dramatically decreased.

The Fenton and Fenton-like system enhancing agent is simple to use and easy to apply. There is no additional treatment equipment requirement with a wide range of applicability while the reaction temperature requirement is low. The quantity requirement of Fenton and Fenton-like system enhancing agent is low. After the Fenton and Fenton-like system enhancing agent is dissolved in the water, the water is safe to human and animals and no additional equipment or steps is required for substance removal.

### Embodiment 6

The embodiment 6 and the embodiment 5 have identical steps and parameters except that in the embodiment 6, the Fenton and Fenton-like system enhancing agent further includes one or more of the followings: ammonium sulfite, sulfite, sodium metabisulfite, lithium metabisulfite, potassium metabisulfite, magnesium metabisulfite, calcium metabisulfite, ammonium metabisulfite, sodium dithionite, lithium dithionite, potassium dithionite, magnesium dithionite, calcium dithionite, ammonium dithionite, humic substance, isopropyl hydroxylamine and potassium permanganate.

### Embodiment 7

The embodiment 7 and the embodiment 5 or 6 have identical steps and parameters except that in the embodiment 7, the molar ratio of the Fenton and Fenton-like system enhancing agent being added and the pollutants in the water subject to treatment is 5˜10:1˜100, the molar ratio of the hydrogen peroxide being added and the pollutants in the water subject to treatment is 2˜50:1, and the molar ratio of the agent for enhancement being added and the pollutants in the water subject to treatment is 0.2˜0.8:1.

### Embodiment 8

The embodiment 8 and the embodiment 5 or 6 have identical steps and parameters except that in the embodiment 8, the molar ratio of the Fenton and Fenton-like system enhancing agent being added and the pollutants in the water subject to treatment is 10:1˜10, the molar ratio of the hydrogen peroxide being added and the pollutants in the water subject to treatment is 3˜20:1, and the molar ratio of the agent for enhancement being added and the pollutants in the water subject to treatment is 0.3˜0.7:1.

### Embodiment 9

The embodiment 9 and the embodiment 5 or 6 have identical steps and parameters except that in the embodiment 9, the molar ratio of the Fenton and Fenton-like system enhancing agent being added and the pollutants in the water subject to treatment is 10:1, the molar ratio of the hydrogen peroxide being added and the pollutants in the water subject to treatment is 10:1, and the molar ratio of the agent for enhancement being added and the pollutants in the water subject to treatment is 0.25:1.

### Embodiment 10

The embodiment 10 and the embodiment 5 or 6 have identical steps and parameters except that in the embodiment 10, further comprising the step of: applying UV radiation, microwave radiation, ultrasonic oscillation, external electric or magnetic field during the process of enhancement treatment of the Fenton or Fenton-like system.

### Embodiment 11

The embodiment 11 and the embodiment 5, 6 or 10 have identical steps and parameters except that in the embodiment 11, further comprising the step of: introducing air, oxygen gas or ozone for aeration during the process of enhancement treatment of the Fenton or Fenton-like system.

### Embodiment 12

The Fenton and Fenton-like system enhancing agent is used by employing the following method of use which comprises the steps of: adding a Fenton and Fenton-like system enhancing agent, an agent for enhancement and hydrogen peroxide into water subject to treatment, wherein the water subject to treatment contains pollutants; and mixing uniformly and allowing reaction such that an enhancement treatment for the Fenton and Fenton-like system is complete, wherein a concentration of benzoic acid, which is a pollutant in the water subject to treatment, is 40 μmol/L, a concentration of hydrogen peroxide is 0.4 mmol/L after the Fenton and Fenton-like enhancing agent and hydrogen peroxide are added into the water subject to treatment, a concentration of the enhancing agent is 0.4 mmol/L, the agent for enhancement is salt of divalent iron, a concentration of the salt of divalent iron in the water subject to treatment is 10 μmol/L, the Fenton and Fenton-like enhancing agent is hydroxylamine.

Because benzoic acid is a target indicator for hydroxyl radical, the water subject to treatment of this embodiment is distilled water with added benzoic acid.

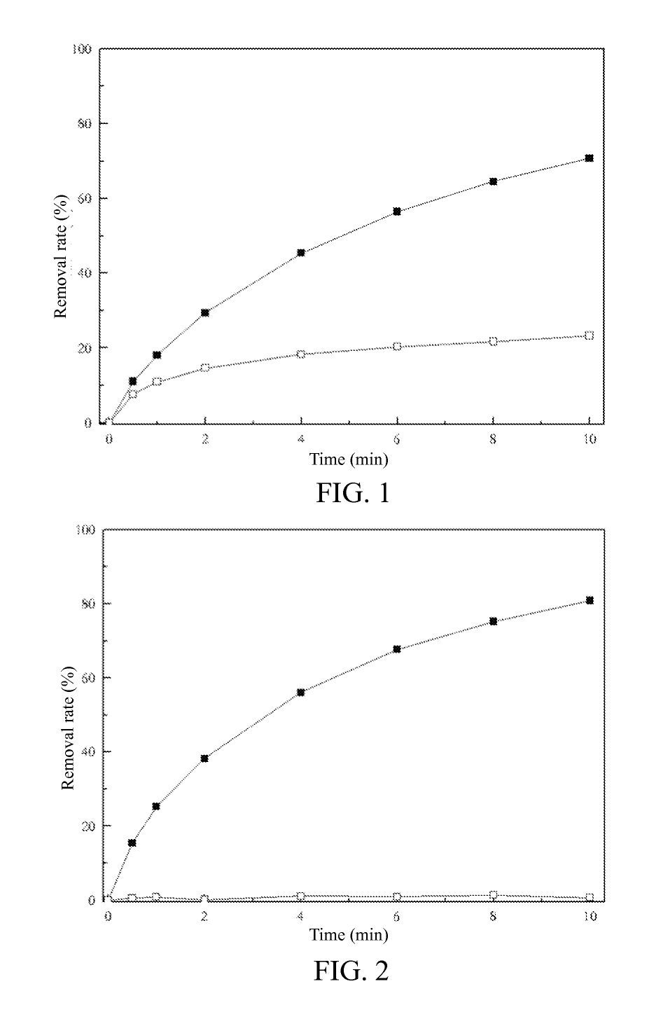

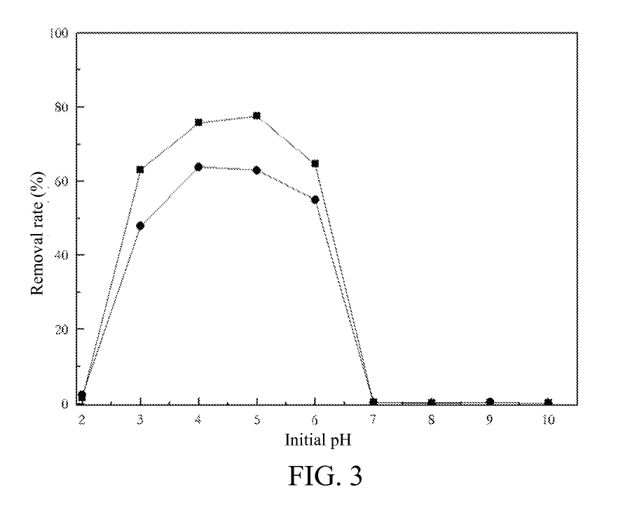

According to this embodiment, the process of enhancement treatment is carried out at 17° C. for 10 minutes. A standard Fenton system is used as a control (in which all steps and conditions are the same as the testing group except that no Fenton or Fenton-like enhancing agent is added and a water pH is adjusted to 3 in the control). The testing results of enhancement treatment of water are shown in FIG. 1 of the drawings. The testing results demonstrate that the removal rate of pollutant (benzoic acid) by standard Fenton system is only 20% at 10 min while the removal rate of pollutant (benzoic acid) by the Fenton system with enhancement treatment of the present invention is 71.7% at 10 min, which is much higher than that of the standard Fenton system. In order to achieve the same removal rate of the Fenton system with enhancement treatment of the present invention by using standard Fenton system, the amount of the agent for enhancement (salt of divalent iron) has to be increased by 3˜5 times. In addition, this embodiment of the present invention greatly increases the oxidant utilization and increases the rate of hydroxyl radical generation.

Through employing the steps and parameters of this embodiment of the present invention, the required water quality standard can be achieved with an extremely low amount of agent for enhancement. Accordingly, because the level of agent for enhancement is very low, it is not necessary to remove the agent for enhancement in the water after treatment. During application of this embodiment of the present invention, if the water subject to treatment contains a certain amount of Fe²⁺ (such as ground water and surface water), the amount of agent for enhancement can be further reduced and the cost of water treatment is further decreased.

According to this embodiment of the present invention, the Fenton and Fenton-like system enhancing agent does not require any adjustment of water pH for carrying out the Fenton or Fenton-like reaction during the enhancement treatment.

### Embodiment 13

The Fenton and Fenton-like system enhancing agent is used by employing the following method of use which comprises the steps of: adding a Fenton and Fenton-like system enhancing agent, an agent for enhancement and hydrogen peroxide into water subject to treatment, wherein the water subject to treatment contains pollutants; and mixing uniformly and allowing reaction such that an enhancement treatment for the Fenton and Fenton-like system is complete, wherein a concentration of benzoic acid, which is a pollutant in the water subject to treatment, is 40 μmol/L, a concentration of hydrogen peroxide is 0.4 mmol/L after the Fenton and Fenton-like enhancing agent and hydrogen peroxide are added into the water subject to treatment, a concentration of the enhancing agent is 0.4 mmol/L, the agent for enhancement is salt of trivalent iron, a concentration of the salt of trivalent iron in the water subject to treatment is 10 μmol/L, the Fenton and Fenton-like enhancing agent is hydroxylamine sulfate.

According to this embodiment, the process of enhancement treatment is carried out at 17° C. for 10 minutes. A standard Fenton system is used as a control (in which all steps and conditions are the same as the testing group except that no Fenton or Fenton-like enhancing agent is added and a water pH is adjusted to 3 in the control). The testing results of enhancement treatment of water are shown in FIG. 2 of the drawings. The testing results demonstrate that the removal rate of pollutant (benzoic acid) by standard Fenton system almost remains unchanged at 10 min while the removal rate of pollutant (benzoic acid) by the Fenton system with enhancement treatment of the present invention is 81.5% at 10 min, which is higher than that of the standard Fenton system.

Because the cost of trivalent iron salt is lower than that of divalent iron salt and divalent iron salt is extremely easily oxidized during transport and reaction, the Fenton and Fenton-like enhancing agent according to this embodiment of the present invention provides further advantageous practical effect. In addition, this embodiment of the present invention greatly increases the oxidant utilization and increases the rate of hydroxyl radical generation, thereby increasing the rate of Fenton system water treatment.

Through employing the steps and parameters of this embodiment of the present invention, the required water quality standard can be achieved with an extremely low amount of agent for enhancement. Accordingly, because the level of agent for enhancement is very low, it is not necessary to remove the agent for enhancement in the water after treatment. During application of this embodiment of the present invention, if the water subject to treatment contains a certain amount of Fe²⁺ (such as ground water and surface water), the amount of agent for enhancement can be further reduced and the cost of water treatment is further decreased.

According to this embodiment of the present invention, the Fenton and Fenton-like system enhancing agent does not require any adjustment of water pH for carrying out the Fenton or Fenton-like system enhancement treatment.

### Embodiment 14

The embodiment 14 and the embodiment 12 have identical steps and parameters except that in the embodiment 14, the agent for enhancement is salt of divalent iron and cerium, a molar ratio of salt of divalent iron and cerium is 1:1, the Fenton and Fenton-like system enhancing agent is sodium sulfite, hydroxylamine sulfate and hydroxylamine solution, a molar ratio of sodium sulfite, hydroxylamine sulfate and hydroxylamine (H₂NOH) in the hydroxylamine solution is 2:1:4.

According to this embodiment, the process of enhancement treatment is carried out at 17° C. for 10 minutes. The removal rate of pollutant (benzoic acid) is 73.4%.

### Embodiment 15

The embodiment 15 and the embodiment 12 have identical steps and parameters except that in the embodiment 15, the agent for enhancement is salt of divalent iron, ZrO₂ and zerovalent iron, a molar ratio of salt of divalent iron, ZrO₂ and zerovalent iron is 6:3:1, the Fenton and Fenton-like system enhancing agent is N,N,N′,N′-tetrasubstituted p-phenylenediamine, N-N-diethylhydroxylamine and isopropyl hydroxylamine, a molar ratio of N,N,N′,N′-tetrasubstituted p-phenylenediamine, N-N-diethylhydroxylamine and isopropyl hydroxylamine is 2:2:1.

According to this embodiment, the process of enhancement treatment is carried out at 17° C. for 10 minutes. The removal rate of pollutant (benzoic acid) is 73.4%.

### Embodiment 16

The embodiment 16 and the embodiment 12 have identical steps and parameters except that in the embodiment 16, the agent for enhancement is iron (II) oxide, the Fenton and Fenton-like system enhancing agent is hydroxylamine perchlorate and calcium sulfite, a molar ratio of hydroxylamine perchlorate and calcium sulfite is 6:1.

According to this embodiment, the process of enhancement treatment is carried out at 20° C. for 10 minutes. The removal rate of pollutant (benzoic acid) is 71.5%.

### Embodiment 17

The embodiment 17 and the embodiment 12 have identical steps and parameters except that in the embodiment 17, the agent for enhancement is iron (III) oxide, salt of divalent iron, iridium and CoO, a molar ratio of iron (III) oxide, salt of divalent iron, iridium and CoO is 1:3:1:1, the Fenton and Fenton-like system enhancing agent is amino ethanolamine.

According to this embodiment, the process of enhancement treatment is carried out at 17° C. for 12 minutes. The removal rate of pollutant (benzoic acid) is 76.4%.

### Embodiment 18

The embodiment 18 and the embodiment 12 have identical steps and parameters except that in the embodiment 18, the agent for enhancement is iron (II) oxide, Fe4O3 and Iron (III) oxide-hydroxide, a molar ratio of iron (II) oxide, Fe4O3 and Iron (III) oxide-hydroxide is 4:1:2, the Fenton and Fenton-like system enhancing agent is magnesium sulfite.

According to this embodiment, the process of enhancement treatment is carried out at 17° C. for 12 minutes. The removal rate of pollutant (benzoic acid) is 77.9%.

As shown from the above embodiments 12, 14, 15, 16, 17 and 18, if the ratio of the amount of the Fenton and Fenton-like enhancing agent, the agent for enhancement and the hydrogen peroxide being added remains unchanged, each of the Fenton systems with enhancement treatment has similar removal effect. This shows that the components of the Fenton and Fenton-like enhancing agent are not fixed and can be freely adjusted for achieving the enhancement treatment with excellent removal effect and widening the range of applicability of the Fenton reagent. In addition, the water temperature has no significant effect on the removal rate. On the other hand, as the treatment time is increased, the removal rate is also increased.

### Embodiment 19

The embodiment 19 and the embodiment 13 have identical steps and parameters except that in the embodiment 19, the agent for enhancement is iron (III) oxide, salt of trivalent iron and osmium, a molar ratio of iron (III) oxide, salt of trivalent iron and osmium is 4:4:3, the Fenton and Fenton-like enhancing agent is sodium sulfite and hydroxylamine solution, a molar ratio of sodium sulfite and hydroxylamine (H₂NOH) in the hydroxylamine solution is 2:1.

According to this embodiment, the process of enhancement treatment is carried out at 17° C. for 10 minutes. The removal rate of pollutant (benzoic acid) is 80.8%.

### Embodiment 20

The embodiment 20 and the embodiment 13 have identical steps and parameters except that in the embodiment 20, the agent for enhancement is iron (III) oxide, salt of trivalent iron, yttrium and Rh₂O₃.5(H₂O), a molar ratio of iron (III) oxide, salt of trivalent iron, yttrium and Rh₂O₃.5(H₂O) is 4:2:1:1, the Fenton and Fenton-like enhancing agent is magnesium sulfite.

According to this embodiment, the process of enhancement treatment is carried out at 17° C. for 10 minutes. The removal rate of pollutant (benzoic acid) is 82.1%.

### Embodiment 21

The embodiment 21 and the embodiment 13 have identical steps and parameters except that in the embodiment 21, the agent for enhancement is molybdenum, tungsten, Nb₂O₅ and V₂O₅, a molar ratio of molybdenum, tungsten, Nb₂O₅ and V₂O₅ is 1:1:1:1, the Fenton and Fenton-like enhancing agent is sodium sulfite and lithium sulfite, a molar ratio of sodium sulfite and lithium sulfite is 2:1.

According to this embodiment, the process of enhancement treatment is carried out at 17° C. for 10 minutes. The removal rate of pollutant (benzoic acid) is 80.8%.

### Embodiment 22

The embodiment 22 and the embodiment 13 have identical steps and parameters except that in the embodiment 22, the agent for enhancement is IrO₂, RuO₂ and Rh₂O₃, a molar ratio of IrO₂, RuO₂ and Rh₂O₃ is 2:1:2, the Fenton and Fenton-like enhancing agent is magnesium sulfite, hydroxylamine sulfate and isopropyl hydroxylamine, a molar ratio of magnesium sulfite, hydroxylamine sulfate and isopropyl hydroxylamine is 1:1:1.

According to this embodiment, the process of enhancement treatment is carried out at 17° C. for 10 minutes. The removal rate of pollutant (benzoic acid) is 82.5%.

### Embodiment 23

The embodiment 23 and the embodiment 13 have identical steps and parameters except that in the embodiment 23, the agent for enhancement is zirconium, niobium and molybdenum, a molar ratio of zirconium, niobium and molybdenum is 1:1:3, the Fenton and Fenton-like enhancing agent is lithium sulfite, potassium sulfite and N-N-diethylhydroxylamine, a molar ratio of lithium sulfite, potassium sulfite and N-N-diethylhydroxylamine is 1:2:1.

According to this embodiment, the process of enhancement treatment is carried out at 17° C. for 10 minutes. The removal rate of pollutant (benzoic acid) is 81.7%.

### Embodiment 24

The embodiment 24 and the embodiment 13 have identical steps and parameters except that in the embodiment 24, the agent for enhancement is zerovalent iron, salt of high valent iron, nickel and WO₃, a molar ratio of zerovalent iron, salt of high valent iron, nickel and WO₃ is 1:2:1:3, the Fenton and Fenton-like enhancing agent is hydroxylamine hydrochloride.

According to this embodiment, the process of enhancement treatment is carried out at 17° C. for 10 minutes. The removal rate of pollutant (benzoic acid) is 81.5%.

### Embodiment 25

The embodiment 25 and the embodiment 13 have identical steps and parameters except that in the embodiment 25, the agent for enhancement is Iron (III) oxide-hydroxide and OsO₄, a molar ratio of Iron (III) oxide-hydroxide and OsO₄ is 2:1, the Fenton and Fenton-like enhancing agent is hydrazine and potassium permanganate, a molar ratio of hydrazine and potassium permanganate is 1:1.

According to this embodiment, the process of enhancement treatment is carried out at 25° C. for 10 minutes. The removal rate of pollutant (benzoic acid) is 82.4%.

### Embodiment 26

The embodiment 26 and the embodiment 13 have identical steps and parameters except that in the embodiment 26, the agent for enhancement is Fe4O3, yttrium and iron (III) oxide, a molar ratio of Fe4O3, yttrium and iron (III) oxide is 2:2:3, the Fenton and Fenton-like enhancing agent is magnesium sulfite and hydroxylamine sulfate, a molar ratio of magnesium sulfite and hydroxylamine sulfate is 1:1.

According to this embodiment, the process of enhancement treatment is carried out at 12° C. for 10 minutes. The removal rate of pollutant (benzoic acid) is 80.1%.

### Embodiment 27

The embodiment 27 and the embodiment 13 have identical steps and parameters except that in the embodiment 27, the agent for enhancement is Tc₂O₇ and silver, a molar ratio of Tc₂O₇ and silver is 2:1, the Fenton and Fenton-like enhancing agent is isopropyl hydroxylamine.

According to this embodiment, the process of enhancement treatment is carried out at 17° C. for 12 minutes. The removal rate of pollutant (benzoic acid) is 84.6%.

As shown from the above embodiments 13, 19, 20, 21, 22, 23, 24, 25 and 26, if the ratio of the amount of the Fenton and Fenton-like enhancing agent, the agent for enhancement and the hydrogen peroxide being added remains unchanged, each of the Fenton systems with enhancement treatment has similar removal effect. This shows that the components of the Fenton and Fenton-like enhancing agent are not fixed and can be freely adjusted for achieving the enhancement treatment with excellent removal effect and widening the applicability of the Fenton reagent. In addition, the water temperature has no significant effect on the removal rate. On the other hand, as the treatment time is increased, the removal rate is also increased.

### Embodiment 28

The embodiment 28 employs the technical solutions of embodiment 12 and 13 to carry out the enhancement treatment of the Fenton and Fenton-like system for polluted water having an initial pH ranged from 1˜14 respectively.

The initial pH is the water pH after the Fenton and Fenton-like enhancing agent, the agent for enhancement and the hydrogen peroxide are added.

In this embodiment 28, identical steps and parameters of the embodiment 12 and 13 are used respectively. The testing results of enhancement treatment of water are shown in FIG. 3 of the drawings. According to the testing results, the Fenton and Fenton-like enhancing agent is applicable for enhancement treatment of water if the initial pH is smaller than 7. Since the water pH is lowered by 1˜2 after the agent for enhancement, the hydrogen peroxide and the Fenton and Fenton-like enhancing agent are added, the present invention can be used to treat any polluted water having an original pH smaller than 9. Accordingly, the range of applicable pH of the present invention is significantly wider than Fenton and Fenton-like system and the present invention has a good enhancement treatment effect for polluted water having an initial pH smaller than 7.

### Embodiment 29

The embodiment 29 and the embodiment 12 have identical steps and parameters except that in the embodiment 29, the agent for enhancement is iron (II) oxide, Fe4O3 and iron (III) oxide-hydroxide, a molar ratio of iron (II) oxide, Fe4O3 and iron (III) oxide-hydroxide is 4:1:2, the Fenton and Fenton-like enhancing agent is ammonium metabisulfite, potassium dithionite and magnesium sulfite, a molar ratio of ammonium metabisulfite, potassium dithionite and magnesium sulfite is 1:1:35.

According to this embodiment, the process of enhancement treatment is carried out at 17° C. for 10 minutes. The removal rate of pollutant (benzoic acid) is 72.6%.

### Embodiment 30

The embodiment 30 and the embodiment 13 have identical steps and parameters except that in the embodiment 30, the agent for enhancement is Fe4O3, yttrium and iron (III) oxide, a molar ratio of Fe4O3, yttrium and iron (III) oxide is 2:2:3, the Fenton and Fenton-like enhancing agent is magnesium sulfite, calcium dithionite and hydroxylamine sulfate, a molar ratio of magnesium sulfite, calcium dithionite and hydroxylamine sulfate is 4:5:1.

According to this embodiment, the process of enhancement treatment is carried out at 17° C. for 10 minutes. The removal rate of pollutant (benzoic acid) is 78.5%.

### Embodiment 31

The embodiment 31 and the embodiments 1, 2 and 3 have identical steps and parameters except that in the embodiment 31: the Fenton and Fenton-like enhancing agent further comprises isopropyl hydroxylamine and/or potassium permanganate.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

1. A Fenton and Fenton-like system enhancing agent, characterized in that, the Fenton and Fenton-like system enhancing agent is selected from the group consisting of: sodium sulfite, lithium sulfite, potassium sulfite, magnesium sulfite, calcium sulfite, hydroxylamine hydrochloride, hydroxylamine perchlorate, hydroxylamine sulfate, hydrazine, N-N-diethylhydroxylamine, amino ethanolamine, hydroxylamine solution and N,N,N′,N′-tetrasubstituted p-phenylenediamine.

2. The Fenton and Fenton-like system enhancing agent, as recited in claim 1, characterized in that, wherein the Fenton and Fenton-like system enhancing agent further comprises one or more component selected from the group consisting of: ammonium sulfite, sulfite, sodium metabisulfite, lithium metabisulfite, potassium metabisulfite, magnesium metabisulfite, calcium metabisulfite, ammonium metabisulfite, sodium dithionite, lithium dithionite, potassium dithionite, magnesium dithionite, calcium dithionite, ammonium dithionite and humic substance.

3. The Fenton and Fenton-like system enhancing agent, as recited in claim 1, characterized in that, further comprising isopropyl hydroxylamine, potassium permanganate or a mixture of isopropyl hydroxylamine and potassium permanganate.

4. A method of use of a Fenton and Fenton-like system enhancing agent, characterized in that, said method comprises the steps of: adding a Fenton and Fenton-like system enhancing agent, an agent for enhancement and hydrogen peroxide into water subject to treatment, wherein the water subject to treatment contains pollutants; and mixing uniformly and allowing reaction such that an enhancement treatment of the Fenton and Fenton-like system is complete, wherein a molar ratio of the Fenton and Fenton-like system enhancing agent being added and the pollutants in the water subject to treatment is 1˜10:1˜1000, a molar ratio of the hydrogen peroxide being added and the pollutants in the water subject to treatment is 1˜100:1, and a molar ratio of the agent for enhancement being added and the pollutants in the water subject to treatment is 0.1˜1:1, wherein the agent for enhancement is selected from the group consisting of: salt of divalent iron, salt of trivalent iron, salt of high valent iron, zerovalent iron, Fe₄O₃, Iron (III) oxide, Iron (III) oxide-hydroxide, iron (II) oxide, cobalt, cerium, vanadium, nickel, zinc, chromium, silver, scandium, yttrium, zirconium, niobium, molybdenum, technetium, ruthenium, rhodium, palladium, cadmium, lutetium, hafnium, tungsten, rhenium, osmium, iridium, lawrencium, CoO, Ce₂O₃, CeO₂, V₂O₅, VO₂, NiO, ZnO, Cr₂O₃, Ag₂O, Sc₂O₃, Y₂O₃, ZrO₂, Nb₂O₅, MoO₃, Tc₂O₇, RuO₂, Rh₂O₃, PdO, CdO, Lu₂O₃, HfO₂, WO₃, ReO₂, Re₂O₇, OsO₄, OsO₂, IrO₂ and Rh₂O₃.5(H₂O); wherein the Fenton and Fenton-like system enhancing agent is selected from the group consisting of: sodium sulfite, lithium sulfite, potassium sulfite, magnesium sulfite, calcium sulfite, hydroxylamine hydrochloride, hydroxylamine perchlorate, hydroxylamine sulfate, hydrazine, N-N-diethylhydroxylamine, amino ethanolamine, hydroxylamine solution and N,N,N′,N′-tetrasubstituted p-phenylenediamine.

5. The method of use of a Fenton and Fenton-like system enhancing agent, as recited in claim 4, wherein the molar ratio of the Fenton and Fenton-like system enhancing agent being added and the pollutants in the water subject to treatment is 5˜10:1˜100, the molar ratio of the hydrogen peroxide being added and the pollutants in the water subject to treatment is 2˜50:1, and the molar ratio of the agent for enhancement being added and the pollutants in the water subject to treatment is 0.2˜0.8:1.

6. The method of use of a Fenton and Fenton-like system enhancing agent, as recited in claim 4, wherein the molar ratio of the Fenton and Fenton-like system enhancing agent being added and the pollutants in the water subject to treatment is 10:1˜10, the molar ratio of the hydrogen peroxide being added and the pollutants in the water subject to treatment is 3˜20:1, and the molar ratio of the agent for enhancement being added and the pollutants in the water subject to treatment is 0.3˜0.7:1.

7. The method of use of a Fenton and Fenton-like system enhancing agent, as recited in claim 4, wherein the molar ratio of the Fenton and Fenton-like system enhancing agent being added and the pollutants in the water subject to treatment is 10:1, the molar ratio of the hydrogen peroxide being added and the pollutants in the water subject to treatment is 10:1, and the molar ratio of the agent for enhancement being added and the pollutants in the water subject to treatment is 0.25:1.

8. The method of use of a Fenton and Fenton-like system enhancing agent, as recited in claim 4, wherein the Fenton and Fenton-like system enhancing agent further comprises one or more component selected from the group consisting of: ammonium sulfite, sulfite, sodium metabisulfite, lithium metabisulfite, potassium metabisulfite, magnesium metabisulfite, calcium metabisulfite, ammonium metabisulfite, sodium dithionite, lithium dithionite, potassium dithionite, magnesium dithionite, calcium dithionite, ammonium dithionite and humic substance.

9. The method of use of a Fenton and Fenton-like system enhancing agent, as recited in claim 5, wherein the Fenton and Fenton-like system enhancing agent further comprises one or more component selected from the group consisting of: ammonium sulfite, sulfite, sodium metabisulfite, lithium metabisulfite, potassium metabisulfite, magnesium metabisulfite, calcium metabisulfite, ammonium metabisulfite, sodium dithionite, lithium dithionite, potassium dithionite, magnesium dithionite, calcium dithionite, ammonium dithionite and humic substance.

10. The method of use of a Fenton and Fenton-like system enhancing agent, as recited in claim 6, wherein the Fenton and Fenton-like system enhancing agent further comprises one or more component selected from the group consisting of: ammonium sulfite, sulfite, sodium metabisulfite, lithium metabisulfite, potassium metabisulfite, magnesium metabisulfite, calcium metabisulfite, ammonium metabisulfite, sodium dithionite, lithium dithionite, potassium dithionite, magnesium dithionite, calcium dithionite, ammonium dithionite and humic substance.

11. The method of use of a Fenton and Fenton-like system enhancing agent, as recited in claim 7, wherein the Fenton and Fenton-like system enhancing agent further comprises one or more component selected from the group consisting of: ammonium sulfite, sulfite, sodium metabisulfite, lithium metabisulfite, potassium metabisulfite, magnesium metabisulfite, calcium metabisulfite, ammonium metabisulfite, sodium dithionite, lithium dithionite, potassium dithionite, magnesium dithionite, calcium dithionite, ammonium dithionite and humic substance.

12. The method of use of a Fenton and Fenton-like system enhancing agent, as recited in claim 8, wherein the Fenton and Fenton-like system enhancing agent further comprises one or more component selected from the group consisting of: ammonium sulfite, sulfite, sodium metabisulfite, lithium metabisulfite, potassium metabisulfite, magnesium metabisulfite, calcium metabisulfite, ammonium metabisulfite, sodium dithionite, lithium dithionite, potassium dithionite, magnesium dithionite, calcium dithionite, ammonium dithionite and humic substance.

13. A Fenton and Fenton-like system enhancing agent for Fenton and Fenton-like system, comprising at least one component selected from the group consisting of: sodium sulfite, lithium sulfite, potassium sulfite, magnesium sulfite, calcium sulfite, hydroxylamine hydrochloride, hydroxylamine perchlorate, hydroxylamine sulfate, hydrazine, N-N-diethylhydroxylamine, amino ethanolamine, hydroxylamine solution and N,N,N′,N′-tetrasubstituted p-phenylenediamine, wherein the Fenton and Fenton-like system is arranged for water treatment of polluted water in which a pH of the polluted water has a water pH smaller than 9, thereby a reaction rate of the Fenton and Fenton-like system is increased and an amount of iron ion being added is decreased dramatically such that the polluted water after water treatment contains a level of iron which is capable of meeting a standard safety requirement.

14. The Fenton and Fenton-like system enhancing agent, as recited in claim 13, further comprising at least one component selected from the group consisting of: ammonium sulfite, sulfite, sodium metabisulfite, lithium metabisulfite, potassium metabisulfite, magnesium metabisulfite, calcium metabisulfite, ammonium metabisulfite, sodium dithionite, lithium dithionite, potassium dithionite, magnesium dithionite, calcium dithionite, ammonium dithionite and humic substance.

15. The Fenton and Fenton-like system enhancing agent, as recited in claim 14, further comprising at least one component selected from the group consisting of: isopropyl hydroxylamine and potassium permanganate.
